(19) United States
(12) Patent Application Publication
(10) Pub. No.: US 2004/0017544 A1
(43) Pub. Date: Jan. 29, 2004

(54) Contact lenses and methods for their design

(76) Inventors: Jeffrey H. Roffman, Jacksonville, FL (US); Denwood F. Ross, Austinburg, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/202,323

(22) Filed: Jul. 24, 2002

(51) Int. Cl. A61B 3/00

(52) U.S. Cl. 351/219

(57) ABSTRACT

The present invention provides methods for designing contact lenses and lenses produced by those methods. The optical properties of the lens of the invention provide visual acuity correction for the wearer and, at the same time, compensates for print-through.

### FIELD OF THE INVENTION

The present invention relates to the design and manufacture of ophthalmic lenses. In particular, the invention provides contact lenses in the design of which print-through is taken into account.

### BACKGROUND OF THE INVENTION

The use of contact lenses for the correction of an individual's visual acuity is well known. Conventionally available contact lenses are manufactured using either “hard” or “soft” lens materials. The topography of the cornea on which the contact lens rests is irregular. Thus, when a soft contact lens is on-eye, the irregularities in the topography of the cornea produce irregularities on the back, or eye side, and front, or object side, surface of the lens. The degree to which the corneal irregularities will be reflected on the front surface, or print-through from the back to the front surface, is due in part, to the material used to form the lens. However, the print-through is also due to the corneal irregularities, which are difficult to predict from lens wearer to lens wearer.

It is known to design the back surface of the lens so that it is a substantially duplicate of, and fits, the wearer's corneal topography. These lenses correct the visual defects due to irregularities of the corneal topography, but must maintain perfect translational and rotational alignment on the eye to be effective. Therefore, a need exists for additional lens designs that overcome the changes in optical properties due to corneal topography.

### BRIEF DESCRIPTION OF THE DRAWINGS

FIG. 1 depicts a conventional soft contact lens on a cornea.

FIG. 2 depicts a lens of the invention on a cornea.

### DESCRIPTION OF THE INVENTION AND ITS PREFERRED EMBODIMENTS

The present invention provides methods for designing contact lenses and lenses produced by those methods. The optical properties of the lens of the invention provide visual acuity correction for the wearer and, at the same time, the lens compensates for print-through. In a preferred embodiment, the lenses provide correction for higher order aberrations of the eye.

In one embodiment, the invention provides a method for designing a contact lens comprising, consisting essentially of, and consisting of a.) determining a print-through for a soft contact lens placed on-eye; and b.) designing a front surface for the lens that substantially nullifies the print-through. Additionally, the invention comprises, consists essentially of, and consists of a contact lens having a front and a back surface, wherein the front surface substantially nullifies print-through. By “nullifies” is meant that the effect of the print through of the corneal topography to the lens' front surface is substantially neutralized so that there is little or no effect on the image viewed through the lens.

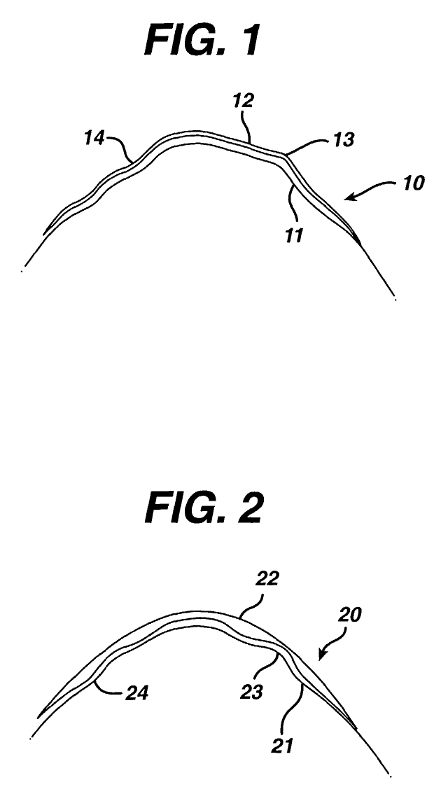

Referring to FIG. 1, a conventional soft contact lens 10 is shown, which depicts the disadvantage of using a conventional soft contact lens on a typical cornea. The cornea 11 has an irregular surface topography and the front surface 12 of lens 10 assumes this irregular shape. This print-through of the corneal topography from the back surface of the lens to the front surface produces elevations and depressions, such as elevation 13 and depression 14 shown in FIG. 1, on the front surface of the lens when the lens is on-eye. These affect the image viewed through the lens by introducing deviations into the front surface geometry. For example, the print-through may cause a front surface that is spherical to reflect the elevations and depressions of the corneal topography thereby altering the spherical shape. It is a discovery of the invention that by measuring these deviations from the desired front surface geometry, a front surface geometry may be provided that substantially nullifies the effect of the print-through on the image viewed through the lens.

This nullification preferably is carried out by providing a peak, or thickened area, on the front surface at the coordinates at which the print-through provides a depression. Similarly, if the print-through induces a peak on the front surface of the lens, the peak will be nullified by a depression, or thinning of the area. Thus, when the lens is on-eye, the front surface will retain the desired geometry and, thus, the optical characteristics, desired. In FIG. 2 is depicted a lens 20 of the invention, which lens is shown on cornea 21. Cornea 21 has an irregular topography as, for example, elevation 23 and depression 24. In lens 20, the irregular corneal topography is nullified by front surface 22.

In the first step of the method of the invention, print-through of the corneal topography onto the front surface of a contact lens is determined. This step may be carried out in any convenient manner. Preferably, a soft lens of the same material, thickness and power as desired for the final lens is placed on-eye and the print-through is measured. Alternatively, the print through may be determined by first measuring the lens' front surface while the lens is off-eye. The lens is then placed on-eye and the front surface is again measured. Print-through may be measured by using a corneal topographer or similar instrument that is capable of determining elevations and their position relative to the corneal apex. Suitable instruments are commercially available.

The data acquired typically will be an elevation map of the front lens surface. In the latter embodiment described above, the print-through may be calculated by computing the difference between the elevation, or sag height, of the lens' front surface off-eye and that elevation as determined on-eye. In the other alternative, the front surface elevation of the lens is simply measured while on-eye.

The elevation data obtained is then used to design the front surface of the final lens. In designing the final lens, for each elevation of the lens surface due to print-through at an x, y coordinate, a depression below the surface at the same coordinate is designed into the lens' front surface that is of substantially the same magnitude as the elevation above the surface. Alternatively, if the measured lens on-eye elevation data shows a peak above the lens surface at a given x, y coordinate, that point on the final lens will have a depression below the surface at the same coordinate, which depression will be of a magnitude equal to that of the peak.

For purposes of forming a lens using the elevation data, the data is transformed mathematically into data on the elevation above and below a mean spherical surface and the elevation data is then transformed into a grid pattern. The grid pattern may be of a rectilinear, polar concentric, or spiral format corresponding to the mechanism by which the lens surface, or a lens mold or insert, may be tooled using a computer numeric controlled lathe, mill, or bit addressable device. Alternatively, the surface may be formed by tooling or lathing using a programmable laser ablation device.

The lenses of the invention may have any of a variety of corrective optical characteristics incorporated onto the front and back surfaces. For example, the lens may have any one or more of spheric, aspheric, bifocal, multifocal, prismatic, or cylindric corrections.

In a more preferred embodiment, the lens provides correction for higher order ocular aberrations, meaning aberrations of the third order or higher. These aberrations may be measured by using ocular optical wavefront measurements carried out using, for example, the output of a crossed cylinder aberroscope, a device that measures ocular Modulation Transfer Function via point spread or line spread, or any similar device which measures, estimates, interpolates or calculates the ocular optical wavefront. The ocular optical wavefront information concerns the optical components of the eye, including the cornea, crystalline lens, system length, tilts, decentrations of the elements of the eye, asymmetrical irregularities, and asphericities. Once measured, the lens is designed to correct for the higher order optical aberrations.

In this customized lens embodiment, conventional sphere-cylindrical prescriptive information also may be used in designing and forming the lens. This information includes the distance sphere, distance astigmatic cylinder power and axis, and the near vision power, if required. This information may be determined using conventional subjective refraction techniques. Alternatively, the sphere, cylinder and axis may be determined based on an analysis of the wavefront accomplished, for example, by reducing the Hartmann-Shack wavefront data to Zernike coefficient terms, and using the relevant terms to derive the sphere, cylinder and axis information.

The lenses designed by the method of the invention are soft contact lenses and may be made using any of the known materials and methods conventionally used in making soft contact lenses. Suitable preferred materials for forming the soft contact lenses of the invention include, without limitation, silicone elastomers, silicone-containing macromers including, without limitation, those disclosed in U.S. Pat. Nos. 5,371,147, 5,314,960, and 5,057,578 incorporated in their entireties herein by reference, hydrogels, silicone-containing hydrogels, and the like and combinations thereof More preferably, the surface is a siloxane, or contains a siloxane functionality, including, without limitation, polydimethyl siloxane macromers, methacryloxypropyl polyalkyl siloxanes, and mixtures thereof, silicone hydrogel or a hydrogel, such as etafilcon A.

What is claimed is:

1. A method for designing a contact lens, comprising the steps of a.) determining a print-through for a soft contact lens placed on-eye; and b.) designing a front surface for the lens that substantially nullifies the lens flexure and print-through.

2. The lens of claim 1, wherein step a.) is carried out by (i.) measuring a front surface of a lens while the lens is off-eye and (ii) measuring, subsequently the front surface of the lens while the lens is on-eye.

3. The lens of claim 1, wherein step a.) is carried out by measuring a front surface of a lens while the lens is the lens is on-eye.

4. The method of claim 1, further comprising providing correction for higher order ocular aberrations.

5. A lens formed by the method of claim 1.

6. A lens formed by the method of claim 1.

7. A lens formed by the method of claim 1.

8. A lens formed by the method of claim 1.
